(19) United States
(12) Patent Application Publication
(10) Pub. No.: US 2018/0161484 A1
(43) Pub. Date: Jun. 14, 2018

(54) SURGICAL IRRIGATION AND SUCTION CONTROL APPARATUS AND CONTROL METHOD THEREOF

(71) Applicant: INTAI TECHNOLOGY CORP., TAICHUNG CITY (TW)

(72) Inventors: Shih-Chang CHUANG, TAICHUNG CITY (TW); Dian-Ying LIN, TAICHUNG CITY (TW)

(21) Appl. No.: 15/376,628

(22) Filed: Dec. 12, 2016

(51) Int. Cl. A61M 1/00; A61M 3/02

(52) U.S. Cl. CPC A61M 1/0062; A61M 1/0064; A61M 1/0035; A61M 1/0086; A61M 3/0279

(57) ABSTRACT

A surgical irrigation and suction control apparatus connecting to an irrigation device and a suction device includes a flexible cannula, a grip, a first tube, a second tube, an irrigation control unit and a suction control unit. The grip is connected to the flexible cannula. The irrigation control unit includes an irrigation switch and an irrigation tube. The irrigation tube is flexible and is connected between the second tube and the irrigation device. The irrigation switch is disposed on the grip and is actuated to change a diameter of the irrigation tube. The suction control unit includes a suction switch and a suction tube. The suction tube is flexible and is connected between the second tube and the suction device. The suction switch is disposed on the grip and is actuated to change a diameter of the suction tube.

### BACKGROUND

### Technical Field

The present disclosure relates to an irrigation and suction control apparatus and an irrigation and suction control method. More particularly, the present disclosure relates to a surgical irrigation and suction control apparatus and a surgical irrigation and suction control method.

### Description of Related Art

During medical procedures, it is common to irrigate or wash a wound with sterilized fluid, such as physiological saline. Furthermore, during surgical procedures, the physician or other health professionals may utilize the irrigation fluid for hydrodissection. In both procedures, the irrigation fluid is provided from a source or a reservoir of fluid of an irrigation device. Commonly, the irrigation fluid is provided under pressure to the surgical site. Furthermore, the physicians and other health professionals also utilize suction to remove spent irrigation fluid, other bodily fluids and debris which may accumulate at the wound site or surgical site. In certain medical procedures, the physician utilizes suction to remove gas. Gas will sometimes hurt the brain tissue at a brain surgical site. Suction, or negative air pressure (some pressure below the ambient pressure), is created by a suction device.

Various kinds of irrigation and suction control apparatuses have been developed in the market. However, they have complex structure and high manufacturing cost. Therefore, it is commercially desirable to develop an irrigation and suction control apparatus and a control method thereof that are easy to use, have low manufacturing cost and can be easily operated by the hand of an operator or a physician.

### SUMMARY

According to one aspect of the present disclosure, a surgical irrigation and suction control apparatus connecting to an irrigation device and a suction device includes a flexible cannula, a grip, a first tube, a second tube, an irrigation control unit and a suction control unit. The flexible cannula includes an inner space. The grip is connected to the flexible cannula and includes an accommodating space communicated with the inner space. The first tube is through the inner space and the accommodating space. The second tube includes a connecting portion. The second tube is through the inner space, and the connecting portion of the second tube is located in the accommodating space. The irrigation control unit includes an irrigation switch and an irrigation tube. The irrigation tube is flexible and is connected between the connecting portion of the second tube and the irrigation device. The irrigation switch is disposed on the grip, and the irrigation switch is actuated to change a diameter of the irrigation tube. The suction control unit includes a suction switch and a suction tube. The suction tube is flexible and is connected between the connecting portion of the second tube and the suction device. The suction switch is disposed on the grip, and the suction switch is to change a diameter of the irrigation tube.

According to another aspect of the present disclosure, a surgical irrigation and suction control apparatus connecting to an irrigation device and a suction device includes a flexible cannula, a grip, a first tube, a second tube, an irrigation control unit, a suction control unit and a partitioning member. The flexible cannula includes an inner space. The grip is connected to the flexible cannula and includes an accommodating space communicated with the inner space. The first tube is through the inner space and the accommodating space. The second tube includes a connecting portion. The second tube is through the inner space, and the connecting portion of the second tube is located in the accommodating space. The irrigation control unit includes an irrigation switch and an irrigation tube. The irrigation tube 620 is flexible. One end of the irrigation tube is connected to the irrigation device. The irrigation switch is disposed on the grip. The irrigation switch is actuated to change a diameter of the irrigation tube. The suction control unit includes a suction switch and a suction tube. The suction tube is flexible, and one end of the suction tube is connected to the suction device. The suction switch is disposed on the grip, and the suction switch is actuated to change a diameter of the irrigation tube. The partitioning member has three terminals which are connected to the connecting portion, the other end of the irrigation tube and the other end of the suction tube, respectively. The irrigation switch and the suction switch are not actuated simultaneously.

According to further another aspect of the present disclosure, a surgical irrigation and suction control method for operating a surgical irrigation and suction control apparatus includes an irrigating step and a sucking step. The irrigating step is for pressing an irrigation switch to increase a diameter of an irrigation tube so as to communicate an irrigation device with a second tube via the irrigation tube. The sucking step is for pressing a suction switch to increase a diameter of a suction tube so as to communicate a suction device with the second tube via the suction tube. The irrigating step and the sucking step are not operated simultaneously.

### BRIEF DESCRIPTION OF THE DRAWINGS

The present disclosure can be more fully understood by reading the following detailed description of the embodiment, with reference made to the accompanying drawings as follows:

FIG. 1 is a schematic view showing a surgical irrigation and suction control apparatus according to one embodiment of the present disclosure;

FIG. 2 is an exploded view showing the surgical irrigation and suction control apparatus of FIG. 1;

FIG. 3 is another exploded view showing the surgical irrigation and suction control apparatus of FIG. 1;

FIG. 4A is a cross-sectional view showing the surgical irrigation and suction control apparatus of FIG. 1;

FIG. 4B is another cross-sectional view showing the surgical irrigation and suction control apparatus of FIG. 1;

FIG. 5 is another schematic view showing the surgical irrigation and suction control apparatus of FIG. 1;

FIG. 6 is a cross-sectional view showing the surgical irrigation and suction control apparatus of FIG. 5;

FIG. 7 is further another schematic view showing the surgical irrigation and suction control apparatus of FIG. 1;

FIG. 8 is a cross-sectional view showing the surgical irrigation and suction control apparatus of FIG. 7;

FIG. 9 is a schematic view showing a surgical irrigation and suction control apparatus according to another embodiment of the present disclosure;

FIG. 10 is a top plan view showing the surgical irrigation and suction control apparatus of FIG. 1;

FIG. 11 is a flow chart showing a surgical irrigation and suction control method according to one embodiment of the present disclosure;

FIG. 12A is a schematic view showing a first tube and a third tube of a surgical irrigation and suction control apparatus according to further another embodiment of the present disclosure;

FIG. 12B is a schematic view showing one end of the surgical irrigation and suction control apparatus of FIG. 12A;

FIG. 12C is a cross-sectional view showing the surgical irrigation and suction control apparatus of FIG. 12A; and

FIG. 12D is a schematic view showing the other end of the surgical irrigation and suction control apparatus of FIG. 12A.

### DETAILED DESCRIPTION

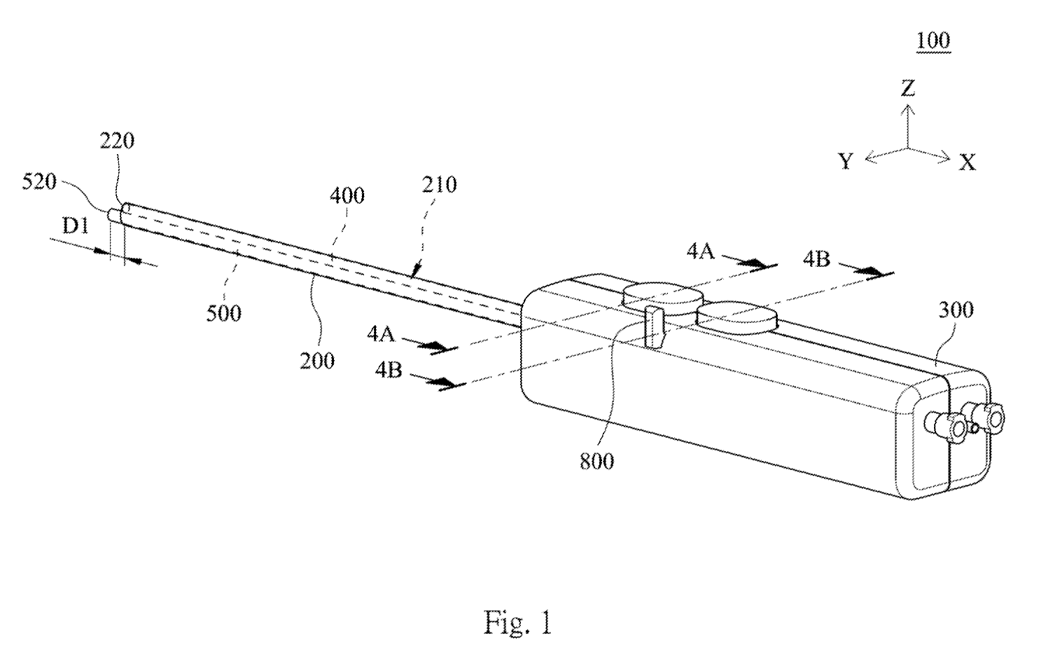

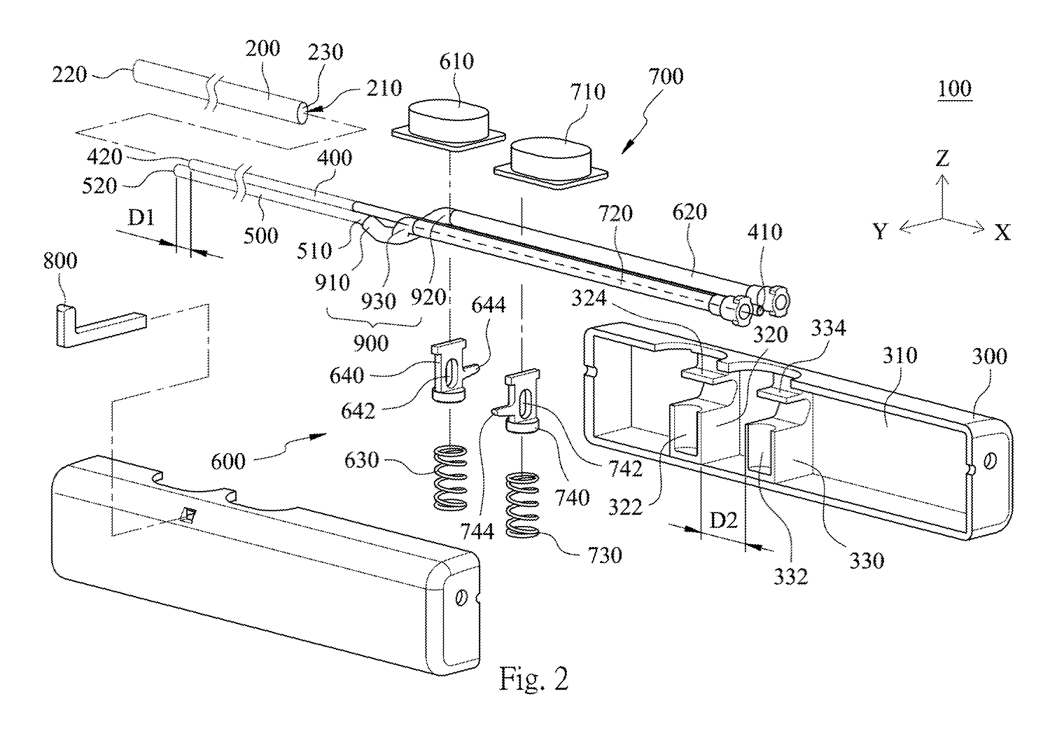

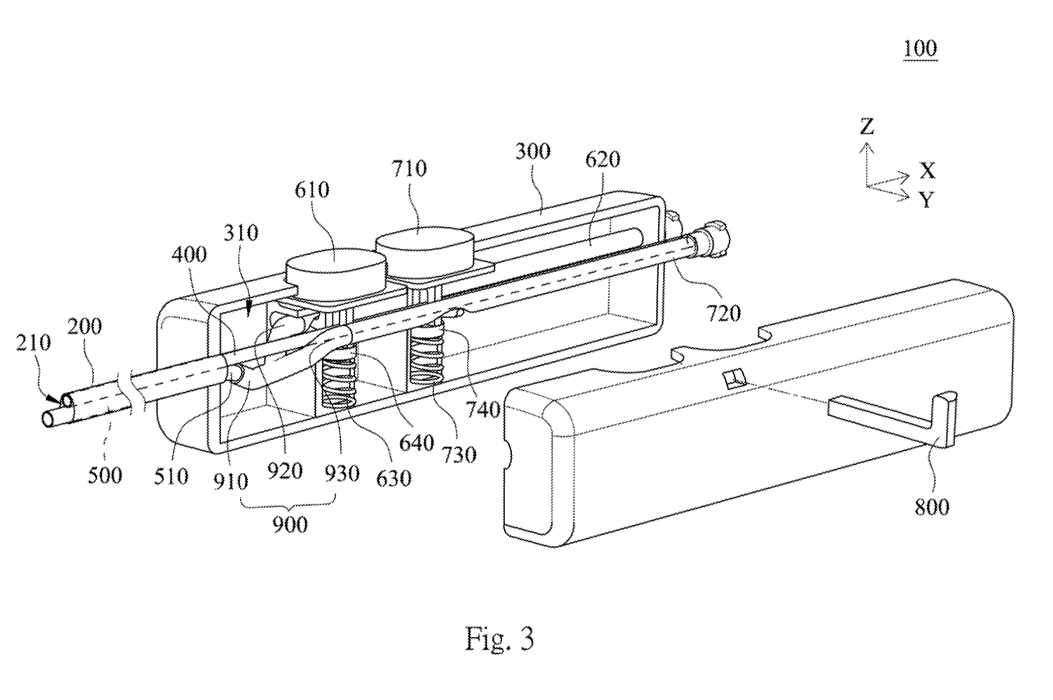

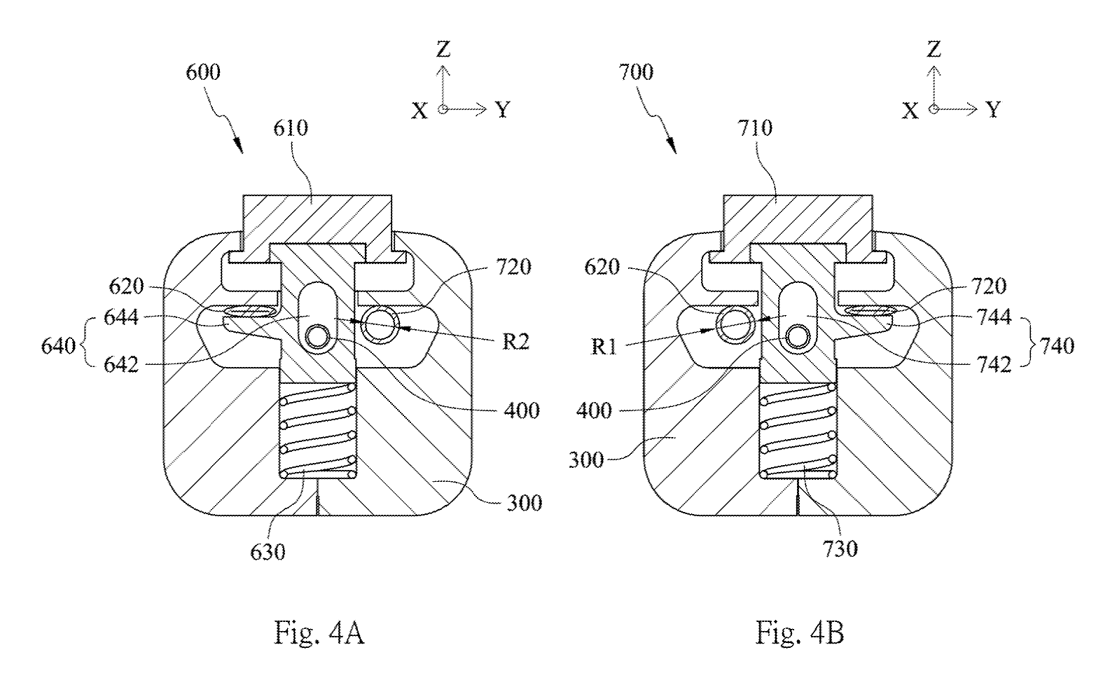

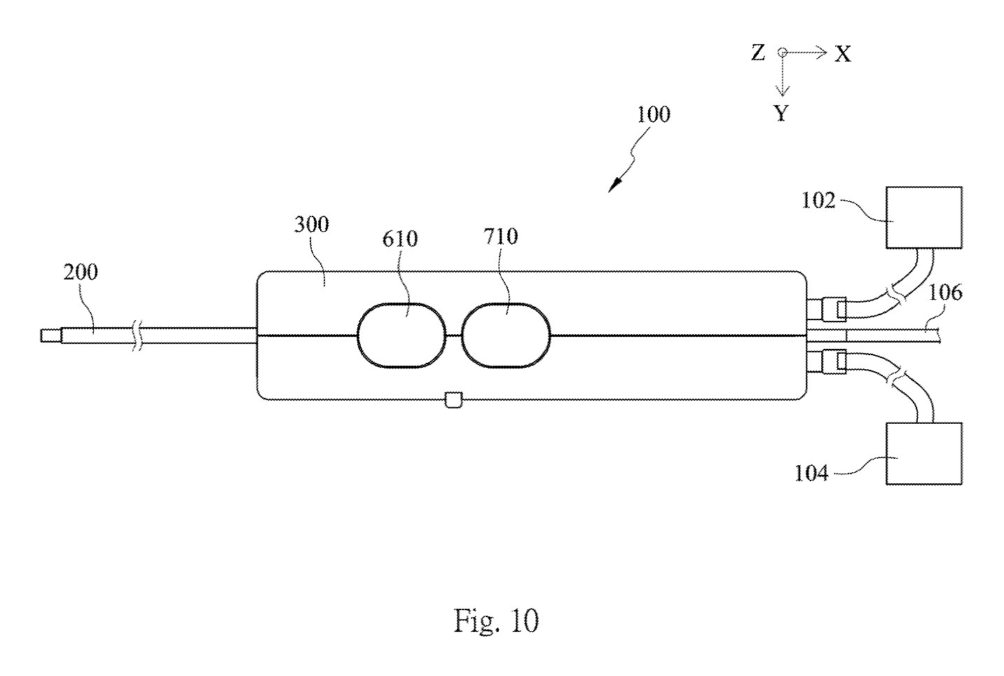

FIG. 1 is a schematic view showing a surgical irrigation and suction control apparatus 100 according to one embodiment of the present disclosure; FIG. 2 is an exploded view showing the surgical irrigation and suction control apparatus 100 of FIG. 1; FIG. 3 is another exploded view showing the surgical irrigation and suction control apparatus 100 of FIG. 1; FIG. 4A is a cross-sectional view showing the surgical irrigation and suction control apparatus 100 of FIG. 1; FIG. 4B is another cross-sectional view showing the surgical irrigation and suction control apparatus 100 of FIG. 1; and FIG. 10 is a top plan view showing the surgical irrigation and suction control apparatus 100 of FIG. 1. The surgical irrigation and suction control apparatus 100 is connected to an irrigation device 102 and a suction device 104. The surgical irrigation and suction control apparatus 100 includes a flexible cannula 200, a grip 300, a first tube 400, a second tube 500, an irrigation control unit 600, a suction control unit 700, a plug 800 and a partitioning member 900.

The flexible cannula 200 includes an inner space 210, a front opening 220 and a connecting end 230. The front opening 220 and the connecting end 230 are located at opposite ends of the flexible cannula 200. The flexible cannula 200 is in a hollow cylindrical shape. The flexible cannula 200 is flexible and can be curved by a force.

The grip 300 is connected to the connecting end 230 of the flexible cannula 200. The grip 300 includes an accommodating space 310, a first positioning seat 320 and a second positioning seat 330. The accommodating space 310 is communicated with the inner space 210. The first positioning seat 320 is disposed in the accommodating space 310. The first positioning seat 320 includes a first positioning slot 322 and a first supporting portion 324, and the first positioning slot 322 is in a cylindrical shape. In addition, the second positioning seat 330 is disposed in the accommodating space 310. The second positioning seat 330 includes a second positioning slot 332 and a second supporting portion 334, and the second positioning slot 332 is in a cylindrical shape. The first positioning seat 320 is parallel to the second positioning seat 330, and the first positioning seat 320 and the second positioning seat 330 are separated by a second distance D2.

The first tube 400 is through the inner space 210 and the accommodating space 310. The endoscope 106 is disposed through the first tube 400. The first tube 400 includes a proximal opening 410 and a first distal opening 420. The first distal opening 420 is aligned to the front opening 220 of the flexible cannula 200. The endoscope 106 may be penetrated from the proximal opening 410 to the first distal opening 420. In the present disclosure, the flexible cannula 200, the first tube 400 and the second tube 500 are made from flexible material and can be curved, so that the operating range of the surgical irrigation and suction control apparatus 100 can be increased in the human body.

The second tube 500 includes a connecting portion 510 and a second distal opening 520. The connecting portion 510 and the second distal opening 520 are located at opposite ends of the second tube 500. The second tube 500 is through the inner space 210, and the connecting portion 510 of the second tube 500 is located in the accommodating space 310. The second distal opening 520 and the front opening 220 are separated by a first distance D1.

The irrigation control unit 600 includes an irrigation switch 610, an irrigation tube 620, a first elastic member 630 and a first supporting member 640. The irrigation switch 610 may be a push button. The irrigation switch 610 is disposed on the grip 300. The irrigation tube 620 is flexible, and one end of the irrigation tube 620 is connected to the irrigation device 102. The irrigation switch 610 is actuated to change a diameter R1 of the irrigation tube 620. The first elastic member 630 is disposed in the first positioning slot 322 of the first positioning seat 320. The first elastic member 630 can be a compression spring. The first supporting member 640 is located in the accommodating space 310. Two ends of the first supporting member 640 are connected to the first elastic member 630 and the irrigation switch 610, respectively. The first supporting member 640 has a first hole 642 and a first protruding portion 644. The irrigation tube 620 is located between the first protruding portion 644 and the first supporting portion 324.

The suction control unit 700 includes a suction switch 710, a suction tube 720, a second elastic member 730 and a second supporting member 740. The suction switch 710 may be a push button. The suction switch 710 is disposed on the grip 300, and the suction switch 710 is actuated to change a diameter R2 of the suction tube 720. The suction tube 720 is flexible, and one end of the suction tube 720 is connected to the suction device 104. The second elastic member 730 is disposed in the second positioning slot 332 of the second positioning seat 330. The second elastic member 730 can be a compression spring. The second supporting member 740 is located in the accommodating space 310. Two ends of the second supporting member 740 are connected to the second elastic member 730 and the suction switch 710, respectively. The second supporting member 740 has a second hole 742 and a second protruding portion 744. The first hole 642 is corresponding to the second hole 742, and the first tube 400 is through the first hole 642 and the second hole 742. This structure can effectively reduce the volume of accommodating space 310 and the weight of the grip 300, so that it is easy to take and operate. The suction tube 720 is located between the second protruding portion 744 and the second supporting portion 334. Thus, the surgical irrigation and suction control apparatus 100 has simple structure and can be fabricated by a simple process at low cost. Therefore, it is favorable for mass production and can be easily used for any operator.

The plug 800 is removably connected to the grip 300 for positioning the irrigation switch 610 or the suction switch 710. The plug 800 can be a L-shaped lever and is easily used by the physician or doctor.

The partitioning member 900 includes three terminals 910, 920, 930 which are connected to the connecting portion 510 of the second tube 500, the other end of the irrigation tube 620 and the other end of the suction tube 720, respectively. The partitioning member 900 is a Y-tube. In detail, the partitioning member 900 has a Y shape when viewed from a Z-axis direction and has a curved shape when viewed from a Y-axis direction. The partitioning member 900 is adjacent to the first tube 400. The first tube 400 is located between the terminal 920 and the terminal 930 when viewed from a X-axis direction. In other words, the first tube 400 is located between the irrigation tube 620 and the suction tube 720 when viewed from the X-axis direction.

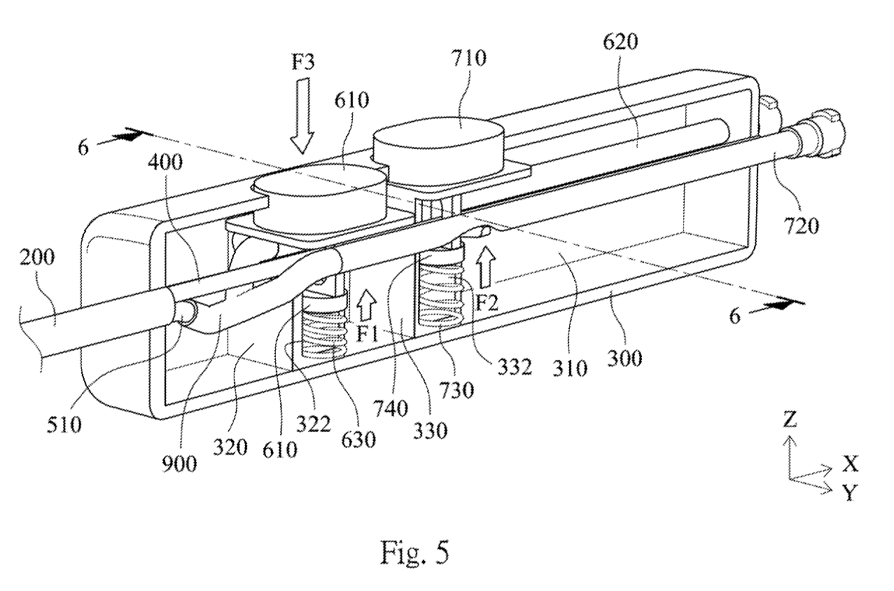

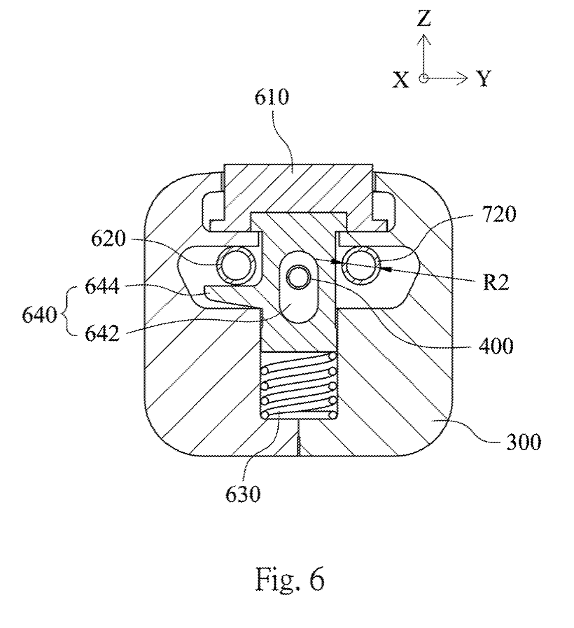

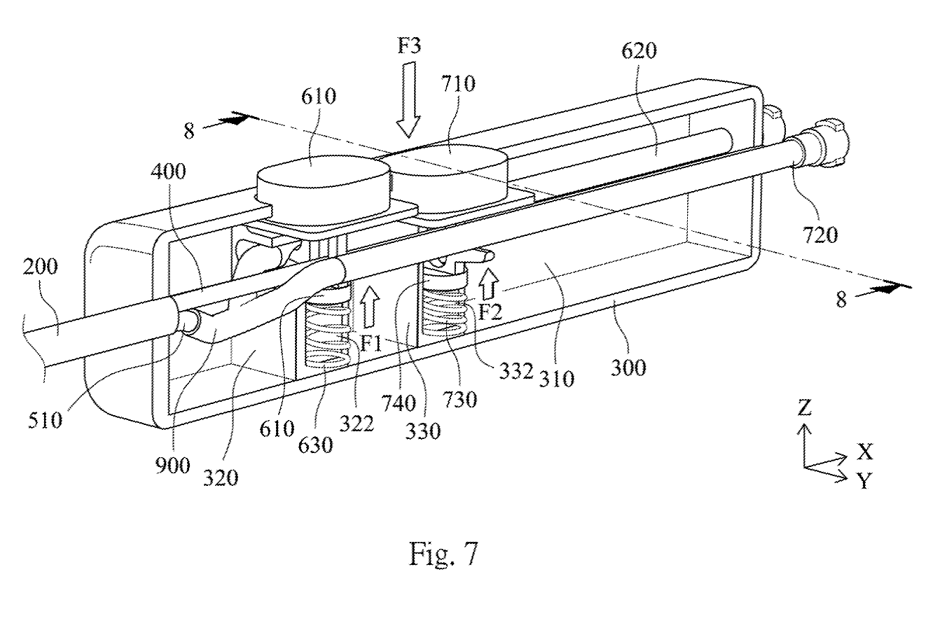

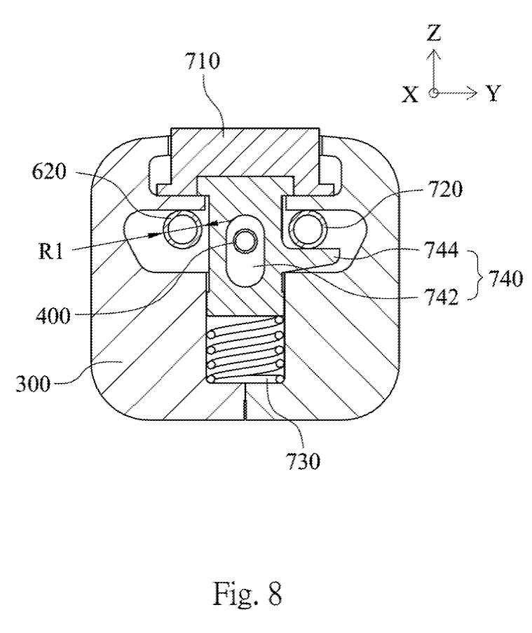

FIG. 5 is another schematic view showing the surgical irrigation and suction control apparatus 100 of FIG. 1; FIG. 6 is a cross-sectional view showing the surgical irrigation and suction control apparatus 100 of FIG. 5; FIG. 7 is further another schematic view showing the surgical irrigation and suction control apparatus 100 of FIG. 1; and FIG. 8 is a cross-sectional view showing the surgical irrigation and suction control apparatus 100 of FIG. 7.

In FIGS. 4A and 7, no external force being exerted on the irrigation switch 610. The first supporting member 640 is pushed by the first restoring force F1 of the first elastic member 630 for flattening the irrigation tube 620 by the first protruding portion 644 of the first supporting member 640 thereby decreasing the diameter R1 of the irrigation tube 620.

In FIGS. 4B and 5, no external force being exerted on the suction switch 710. The second supporting member 740 is pushed by the second restoring force F2 of the second elastic member 730 for flattening the suction tube 720 by the second protruding portion 744 of the second supporting member 740 thereby decreasing the diameter R2 of the suction tube 720.

In FIGS. 5 and 7, the first elastic member 630 is disposed in the first positioning slot 322 of the first positioning seat 320 for applying a first restoring force F1. The second elastic member 730 is disposed in the second positioning slot 332 of the second positioning seat 330 for applying a second restoring force F2.

In FIGS. 5 and 6, the first supporting member 640 is moved in a first direction by exerting an external force F3 provided by the irrigation switch 610. The first direction represents a negative Z-axis direction. The external force F3 given by the physician is greater than the first restoring force F1 given by the first elastic member 630. On the other hand, if the external force F3 is smaller than the first restoring force F1, the first supporting member 640 is moved in a second direction by exerting the first restoring force F1 from the first elastic member 630. The second direction represents a positive Z-axis direction.

In FIGS. 7 and 8, the second supporting member 740 is moved in the first direction by exerting the external force F3 which is provided by the suction switch 710. The external force F3 given by the physician is greater than the second restoring force F2 given by the second elastic member 730. On the other hand, if the external force F3 is smaller than the second restoring force F2, the second supporting member 740 is moved in the second direction by exerting the second restoring force F2 from the second elastic member 730. Most importantly, the irrigation switch 610 and the suction switch 710 are not actuated simultaneously. In other words, irrigation tube 620 and the suction tube 720 are not opened at the same time.

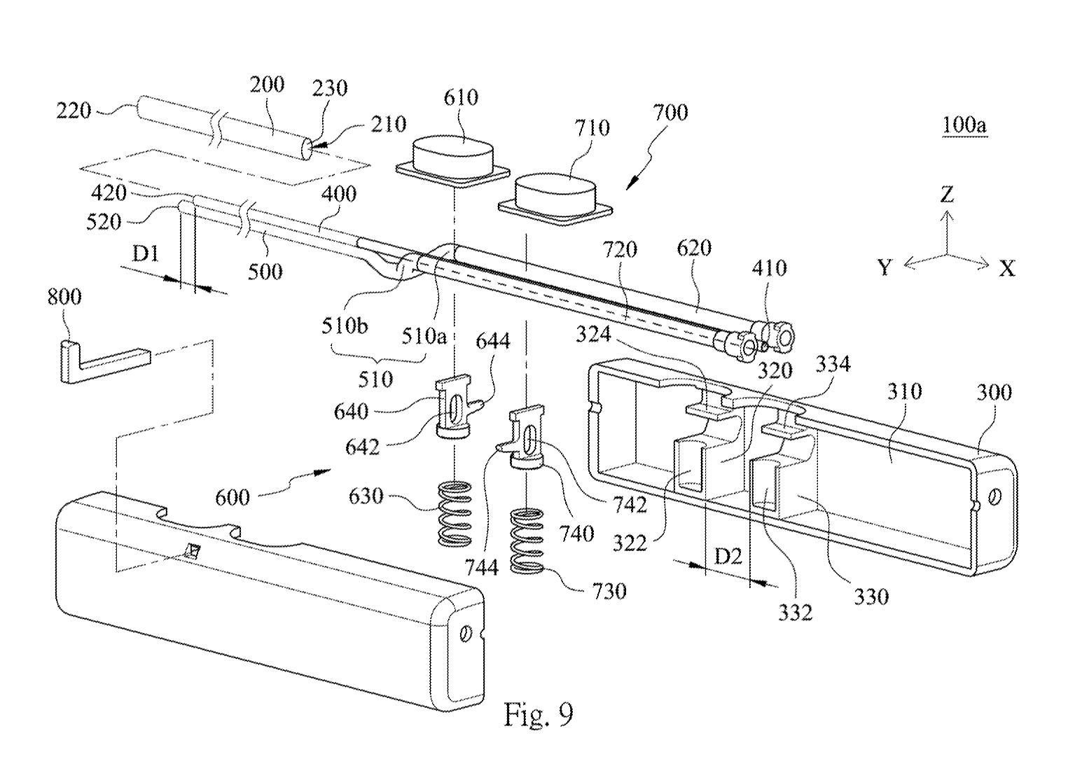

FIG. 9 is a schematic view showing a surgical irrigation and suction control apparatus 100a according to another embodiment of the present disclosure. In FIG. 9, the connecting portion 510 of the second tube 500 has a first opening end 512 and a second opening end 514. The irrigation tube 620 is connected between the first opening end 512 of the connecting portion 510 of the second tube 500 and the irrigation device 102. The suction tube 720 is connected between the second opening end 514 of the connecting portion 510 of the second tube 500 and the suction device 104.

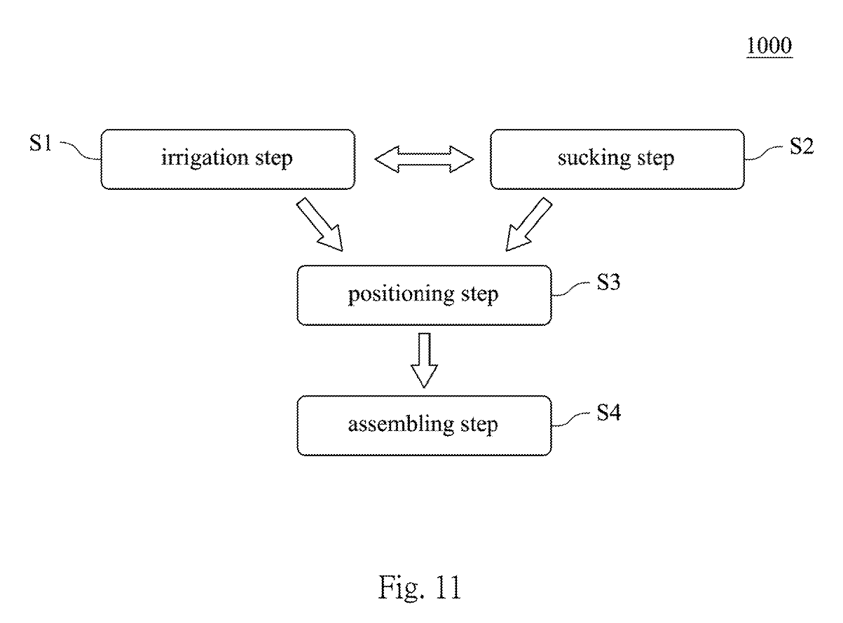

FIG. 11 is a flow chart showing a surgical irrigation and suction control method 1000 according to one embodiment of the present disclosure. The surgical irrigation and suction control method 1000 is for operating the surgical irrigation and suction control apparatus 100, 100a. The surgical irrigation and suction control method 1000 includes an irrigating step S1, a sucking step S2, a positioning step S3 and an assembling step S4. The irrigating step S1 is for pressing an irrigation switch 610 to increase a diameter R1 of an irrigation tube 620 thereby communicating an irrigation device 102 with a second tube 500 via the irrigation tube 620. The sucking step S2 is for pressing a suction switch 710 to increase a diameter R2 of a suction tube 720 thereby communicating a suction device 104 with the second tube 500 via the suction tube 720. The irrigating step S1 and the sucking step S2 are not operated simultaneously. The positioning step S3 is for plugging a plug 800 to the grip 300 to position the irrigation switch 610 or the suction switch 710. The assembling step S4 is for installing an endoscope 106 through the first tube 400. The assembling step S4 can be performed before, during, and/or after the irrigating step S1 or the sucking step S2. By the surgical irrigation and suction control method 1000, irrigation or suction to a surgical site can be effectively performed as well as maintaining clear viewing range through the endoscope 106 during a surgical procedure.

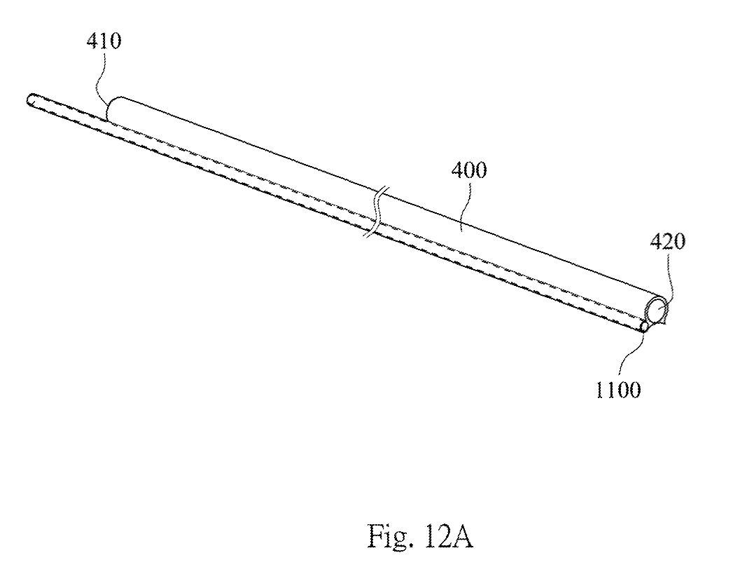

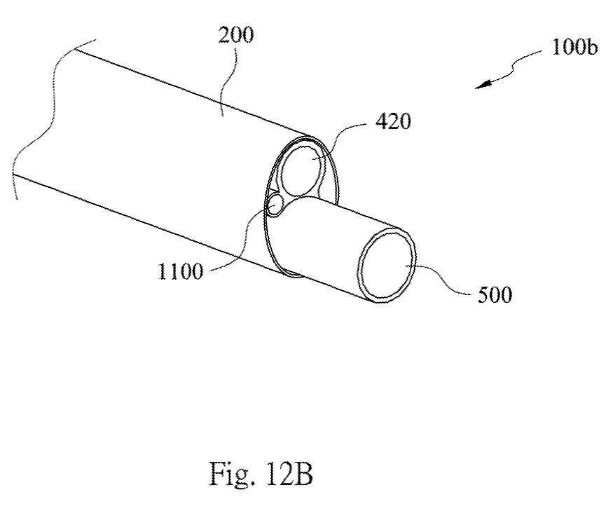

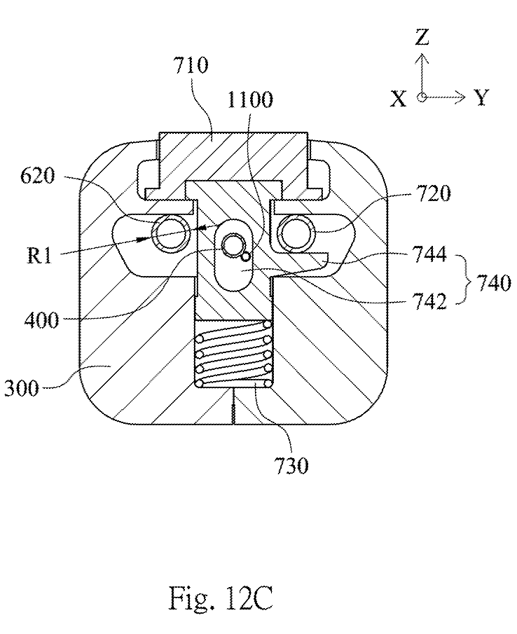

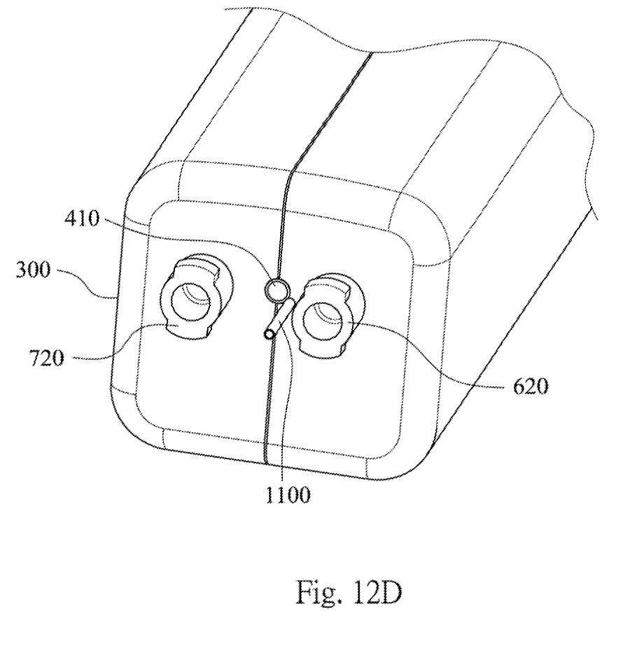

FIG. 12A is a schematic view showing a first tube 400 and a third tube 1100 of a surgical irrigation and suction control apparatus 100b according to further another embodiment of the present disclosure; FIG. 12B is a schematic view showing one end of the surgical irrigation and suction control apparatus 100b of FIG. 12A; FIG. 12C is a cross-sectional view showing the surgical irrigation and suction control apparatus 100b of FIG. 12A; and FIG. 12D is a schematic view showing the other end of the surgical irrigation and suction control apparatus 100b of FIG. 12A. The surgical irrigation and suction control apparatus 100b includes a flexible cannula 200, a grip 300, a first tube 400, a second tube 500, a third tube 1100, an irrigation control unit 600, a suction control unit 700, a plug 800 and a partitioning member 900.

In FIGS. 12A, 12B, 12C and 12D, the details of the flexible cannula 200, a first tube 400, a second tube 500, an irrigation control unit 600, a suction control unit 700, a plug 800 and a partitioning member 900 are the same as the flexible cannula 200, a first tube 400, a second tube 500, an irrigation control unit 600, a suction control unit 700, a plug 800 and a partitioning member 900 of FIG. 2, respectively. In FIGS. 12A, 12B, 12C and 12D, the surgical irrigation and suction control apparatus 100b further includes the third tube 1100 and the grip 300 having an instrument hole. One end of the third tube 1100 is disposed through the instrument hole and is located in the grip 300. The third tube 1100 is connected to the first tube 400. The first tube 400 includes a proximal opening 410 and a first distal opening 420. The third tube 1100 and the first tube 400 are parallel to each other. The other end of the third tube 1100 is disposed in the flexible cannula 200. An opening of the other end of the third tube 1100 and the first distal opening 420 of the first tube 400 are aligned with an opening of the flexible cannula 200. A surgical instrument can be disposed into the third tube 1100 which may be flexible. In addition, the flexible cannula 200, the first tube 400, the second tube 500 and the third tube 1100 may be all flexible for bending. Accordingly, the surgical irrigation and suction control apparatus 100b can be used to simultaneously control the surgical instrument and the endoscope 106 with the functions of surgical irrigation and suction.

According to the aforementioned embodiments and examples, the advantages of the present disclosure are described as follows.

1. The surgical irrigation and suction control apparatus and control method thereof of the present disclosure uses the irrigation control unit and the suction control unit thereby effectively providing irrigation or suction to a surgical site and maintaining clear through the endoscope during a surgical procedure.

2. The surgical irrigation and suction control apparatus and control method thereof of the present disclosure has simple structure and can be fabricated by a simple process at low cost. Therefore, it is favorable for mass production and can be easily used for any operator.

3. The flexible cannula, the first tube and the second tube of the present disclosure are made from flexible material, thus the operating range can be increased on the human body.

4. The volume of accommodating space and the weight of the grip can be effectively reduced, so that it is easy to take and operate.

5. The surgical irrigation and suction control apparatus and control method thereof of the present disclosure uses plural flexible tubes to simultaneously control the surgical instrument and the endoscope with the functions of surgical irrigation and suction.

It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A surgical irrigation and suction control apparatus connecting to an irrigation device and a suction device, the surgical irrigation and suction control apparatus comprising:
a flexible cannula comprising an inner space;
a grip connected to the flexible cannula and comprising an accommodating space communicated with the inner space;
a first tube being through the inner space and the accommodating space;
a second tube comprising a connecting portion, wherein the second tube is through the inner space, and the connecting portion of the second tube is located in the accommodating space;
an irrigation control unit comprising an irrigation switch and an irrigation tube, wherein the irrigation tube is flexible and is connected between the connecting portion of the second tube and the irrigation device, the irrigation switch is disposed on the grip, and the irrigation switch is actuated to change a diameter of the irrigation tube; and
a suction control unit comprising a suction switch and a suction tube, wherein the suction tube is flexible and is connected between the connecting portion of the second tube and the suction device, the suction switch is disposed on the grip, and the suction switch is actuated to change a diameter of the suction tube.

2. The surgical irrigation and suction control apparatus of claim 1, wherein the irrigation switch and the suction switch are not actuated simultaneously.

3. The surgical irrigation and suction control apparatus of claim 1, further comprising:
an endoscope disposed through the first tube.

4. The surgical irrigation and suction control apparatus of claim 3, wherein,
the flexible cannula further comprises a front opening;
the first tube comprises a first distal opening and a proximal opening, and the first distal opening is aligned to the front opening; and
the second tube further comprises a second distal opening, the second distal opening and the front opening are separated by a first distance, and the endoscope is penetrated from the proximal opening to the first distal opening.

5. The surgical irrigation and suction control apparatus of claim 1, wherein the grip further comprising:
a first positioning seat disposed in the accommodating space and comprising a first positioning slot; and
a second positioning seat disposed in the accommodating space and comprising a second positioning slot, wherein the first positioning seat is parallel to the second positioning seat, and the first positioning seat and the second positioning seat are separated by a second distance.

6. The surgical irrigation and suction control apparatus of claim 5, wherein the irrigation control unit further comprising:
a first elastic member disposed in the first positioning slot of the first positioning seat for applying a first restoring force; and
a first supporting member located in the accommodating space, wherein two ends of the first supporting member are connected to the first elastic member and the irrigation switch, respectively;
wherein the first supporting member is moved in a first direction by exerting an external force provided by the irrigation switch, and the first supporting member is moved in a second direction by exerting the first restoring force of the first elastic member.

7. The surgical irrigation and suction control apparatus of claim 6, wherein suction control unit further comprising:
a second elastic member disposed in the second positioning slot of the second positioning seat for applying a second restoring force; and
a second supporting member located in the accommodating space, wherein two ends of the second supporting member are connected to the second elastic member and the suction switch, respectively;
wherein the second supporting member is moved in the first direction by exerting the external force provided by the suction switch, and the second supporting member is moved in the second direction by exerting the second restoring force of the second elastic member.

8. The surgical irrigation and suction control apparatus of claim 7, wherein the first supporting member has a first hole, the second supporting member has a second hole, the first hole is corresponding to the second hole, and the first tube is through the first hole and the second hole.

9. The surgical irrigation and suction control apparatus of claim 7, wherein,
the first supporting member has a first protruding portion, the second supporting member has a second protruding portion, the first positioning seat further comprises a first supporting portion, and the second positioning seat further comprises a second supporting portion;
the irrigation tube is located between the first protruding portion and the first supporting portion, the first supporting member is pushed by the first restoring force of the first elastic member for flattening the irrigation tube by the first protruding portion of the first supporting member thereby decreasing the diameter of the irrigation tube; and
the suction tube is located between the second protruding portion and the second supporting portion, the second supporting member is pushed by the second restoring force of the second elastic member for flattening the suction tube by the second protruding portion of the second supporting member thereby decreasing the diameter of the suction tube.

10. The surgical irrigation and suction control apparatus of claim 1, further comprising:
a plug removably connected to the grip for positioning the irrigation switch or the suction switch.

11. The surgical irrigation and suction control apparatus of claim 1, wherein the first tube is flexible.

12. The surgical irrigation and suction control apparatus of claim 1, wherein the second tube is flexible.

13. A surgical irrigation and suction control apparatus connecting to an irrigation device and a suction device, the surgical irrigation and suction control apparatus comprising:
a flexible cannula comprising an inner space;
a grip connected to the flexible cannula and comprising an accommodating space communicated with the inner space;
a first tube being through the inner space and the accommodating space;
a second tube comprising a connecting portion, wherein the second tube is through the inner space, and the connecting portion of the second tube is located in the accommodating space;
an irrigation control unit comprising an irrigation switch and an irrigation tube, wherein the irrigation tube is flexible, one end of the irrigation tube is connected to the irrigation device, the irrigation switch is disposed on the grip, and the irrigation switch is actuated to change a diameter of the irrigation tube;
a suction control unit comprising a suction switch and a suction tube, wherein the suction tube is flexible, one end of the suction tube is connected to the suction device, the suction switch is disposed on the grip, and the suction switch is actuated to change a diameter of the suction tube; and
a partitioning member having three terminals which are connected to the connecting portion, the other end of the irrigation tube and the other end of the suction tube, respectively;
wherein the irrigation switch and the suction switch are not actuated simultaneously.

14. The surgical irrigation and suction control apparatus of claim 13, wherein the partitioning member is a Y-tube.

15. The surgical irrigation and suction control apparatus of claim 13, further comprising:
an endoscope disposed through the first tube.

16. The surgical irrigation and suction control apparatus of claim 15, wherein,
the flexible cannula further comprises a front opening;
the first tube comprises a first distal opening and a proximal opening, and the first distal opening is aligned to the front opening; and
the second tube further comprises a second distal opening, the second distal opening and the front opening are separated by a first distance, and the endoscope is penetrated from the proximal opening to the first distal opening.

17. The surgical irrigation and suction control apparatus of claim 13, wherein the grip further comprising:
a first positioning seat disposed in the accommodating space and comprising a first positioning slot; and
a second positioning seat disposed in the accommodating space and comprising a second positioning slot, wherein the first positioning seat is parallel to the second positioning seat, and the first positioning seat and the second positioning seat are separated by a second distance.

18. The surgical irrigation and suction control apparatus of claim 17, wherein the irrigation control unit further comprising:
a first elastic member disposed in the first positioning slot of the first positioning seat for applying a first restoring force; and
a first supporting member located in the accommodating space, wherein two ends of the first supporting member are connected to the first elastic member and the irrigation switch, respectively;
wherein the first supporting member is moved in a first direction by exerting an external force provided by the irrigation switch, and the first supporting member is moved in a second direction by exerting the first restoring force of the first elastic member.

19. The surgical irrigation and suction control apparatus of claim 18, wherein suction control unit further comprising:
a second elastic member disposed in the second positioning slot of the second positioning seat for applying a second restoring force; and
a second supporting member located in the accommodating space, wherein two ends of the second supporting member are connected to the second elastic member and the suction switch, respectively;
wherein the second supporting member is moved in the first direction by exerting the external force provided by the suction switch, and the second supporting member is moved in the second direction by exerting the second restoring force of the second elastic member.

20. The surgical irrigation and suction control apparatus of claim 19, wherein the first supporting member has a first hole, the second supporting member has a second hole, the first hole is corresponding to the second hole, and the first tube is through the first hole and the second hole.

21. The surgical irrigation and suction control apparatus of claim 19, wherein,
the first supporting member has a first protruding portion, the second supporting member has a second protruding portion, the first positioning seat further comprises a first supporting portion, and the second positioning seat further comprises a second supporting portion;
the irrigation tube is located between the first protruding portion and the first supporting portion, the first supporting member is pushed by the first restoring force of the first elastic member for flattening the irrigation tube by the first protruding portion of the first supporting member thereby decreasing the diameter of the irrigation tube; and
the suction tube is located between the second protruding portion and the second supporting portion, the second supporting member is pushed by the second restoring force of the second elastic member for flattening the suction tube by the second protruding portion of the second supporting member thereby decreasing the diameter of the suction tube.

22. The surgical irrigation and suction control apparatus of claim 13, further comprising:
a plug removably connected to the grip for positioning the irrigation switch or the suction switch.

23. The surgical irrigation and suction control apparatus of claim 13, wherein the first tube is flexible.

24. The surgical irrigation and suction control apparatus of claim 13, wherein the second tube is flexible.

25. A method for operating a surgical irrigation and suction control apparatus, the method comprising:
an irrigating step for pressing an irrigation switch to increase a diameter of an irrigation tube so as to communicate an irrigation device with a second tube via the irrigation tube; and
a sucking step for pressing a suction switch to increase a diameter of a suction tube so as to communicate a suction device with the second tube via the suction tube;
wherein the irrigating step and the sucking step are not operated simultaneously.

26. The surgical irrigation and suction control method of claim 25, further comprising:
a positioning step for plugging a plug to the grip to position the irrigation switch or the suction switch.

27. The surgical irrigation and suction control method of claim 25, further comprising:
an assembling step for installing an endoscope through the first tube.
